(19) United States
(12) Patent Application Publication
(10) Pub. No.: US 2021/0038877 A1
(43) Pub. Date: Feb. 11, 2021

(54) INTRAVASCULAR PORT BASED SENSING AND TREATMENT SYSTEM

(71) Applicant: ADMETSYS CORPORATION, BOSTON, MA (US)

(72) Inventors: Timothy Valk, Boston, MA (US); Jeffrey Valk, Boston, MA (US); Carol McMorrow, Boston, MA (US)

(21) Appl. No.: 16/976,102

(22) PCT Filed: Mar. 11, 2019
(86) PCT No.: PCT/US19/21550

§ 371 Date: Aug. 27, 2020

Related U.S. Application Data
(60) Provisional application No. 62/641,727, filed on Mar. 12, 2018.

(51) Int. Cl. A61M 39/02

(52) U.S. Cl. CPC A61M 39/0208; A61M 2039/0223; A61M 5/14276; A61M 2205/3553; A61M 2205/0205; A61M 2205/52

(57) ABSTRACT

An intravascular port is provided. The intravascular port includes a housing having an internal chamber and an outer body having a membrane on a surface thereof, the housing including one or more access openings that communicate with the internal chamber. One or more sensors are received within the one or more access openings for sensing one or more physiological parameters of a patient. One or more treatment units are received within the internal chamber of the intravascular port for providing a treatment protocol including medications to the patient. A control unit is in communication with the one or more sensors and the one or more treatment units and is configured to receive a signal from the one or more sensors and output a treatment protocol to the patient.

### FIELD OF THE INVENTION

This invention relates generally to the field of intravascular port based sensing and treatment.

### BACKGROUND OF THE INVENTION

Intravascular ports are known and have been placed in thoracic fluid passageways, such as the vena cava and subclavian vein. The ports are typically implanted and left in place for years and used primarily for infusion of antibiotics and chemotherapy. Each port includes a chamber placed under the skin. The chamber includes a lumen attached distally that accesses a vein. One or more ports can be placed in close proximity and have separate lumens that both access the vein at the same or different sites. In other cases vascular shunts such as arterial-venous shunts have been used for access to a patient's vasculature via catheters as well as ports The ports are accessed through the skin by using an introducer through a port membrane which is adjacent to the subcutaneous tissue and skin.

However, intravascular ports as described above are limited in their use. What is needed, therefore, are ultra-vascular ports for insertion of devices that allow sensing of physiological parameters, provide signal feedback to a control unit and provide a recommended treatment into the accessed vascular space.

### BRIEF SUMMARY OF THE INVENTION

An intravascular port provided. The intravascular port includes a housing having an internal chamber and an outer body having a membrane on a surface thereof, the housing including one or more access openings that communicate with the internal chamber. One or more sensors are received within the one or more access openings for sensing one or more physiological parameters of a patient. One or snore treatment units are received within the internal chamber of the intravascular port for providing a treatment protocol including medications to the patient. A control unit is in communication with the one or more sensors and the one or more treatment units and is configured to receive a signal from the one or more sensors and output a treatment protocol to the patient.

### BRIEF DESCRIPTION OF THE DRAWINGS

For a better understanding of the invention, and to show how the same may be carried into effect, reference will now be made, by way of example, to the accompanying drawings, in which:

FIG. 1 is a perspective view of the intravascular port based sensing device accordance with the invention.

FIG. 2 is a perspective view of the intravascular port of FIG. 1 showing an inserter penetrating a patient's skin and to place a sensor into the port.

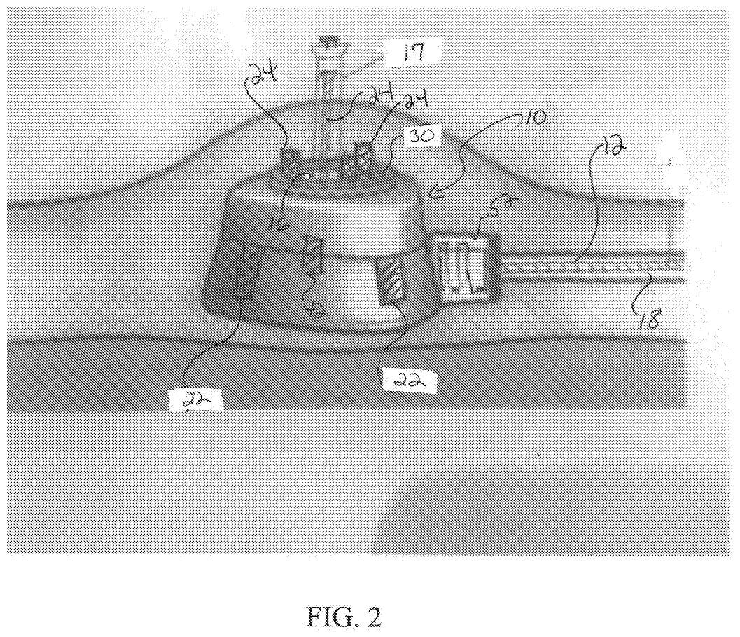

FIG. 3 is a perspective v of an intravascular port in accordance with the invention showing a plurality of sensors and treatment devices place in the port.

FIG. 4 is an schematic illustration of a control unit for use with the intravascular port in accordance with the invention.

FIG. 5 is a flow diagrams depicting sensing and treatment integration.

### DETAILED DESCRIPTION OF THE INVENTION

The system in accordance with the on broadly includes an implantable port housing one or more sensors and a treatment unit. A control unit in communication with the one or more sensors and one or more treatment units may house the sensors and treatment units or be located remotely.

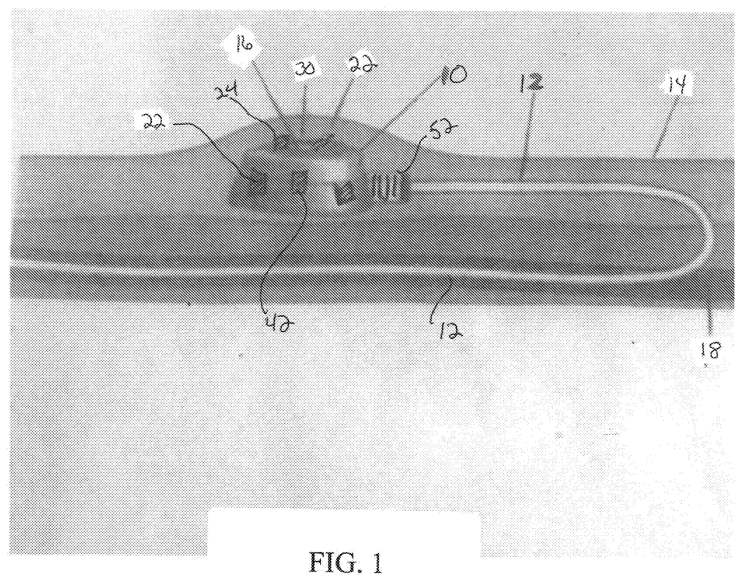

Referring now to FIG. 1 the implantable intravascular port 10 in accordance with the invention is shown. Port includes upper portion 26, lower portion 28 and membrane 30, which is substantially centered on the upper portion 26 and configured to provide access to internal chamber 16. The port is coupled to a catheter 12 and is implanted under the skin 14. The catheter 12 extends from an inters al chamber 16 of the port and into a patient's vasculature 18, such as a vein, artery or vascular shunt, which allows for the sensing and treatment units to measure physiological parameters direct from the patient's blood. Internal chamber 16 is structured as a cavity or lumen, and may include a plurality of chambers or lumens, and couples directly to lumen 20 of catheter 12. Multiple implanted intravascular ports may be aligned to allow a single catheter to be used to access all or some of the ports 10. Intravascular port 10 and catheter 12 may be made of biocompatible materials known to those of skill in the art and coated with antimicrobial material such as silver or antibiotics or anti-clotting material such as polyethylene glycol or heparin. Those of skill in the art will appreciate that other coatings may also be used to allow easy insertion and removal.

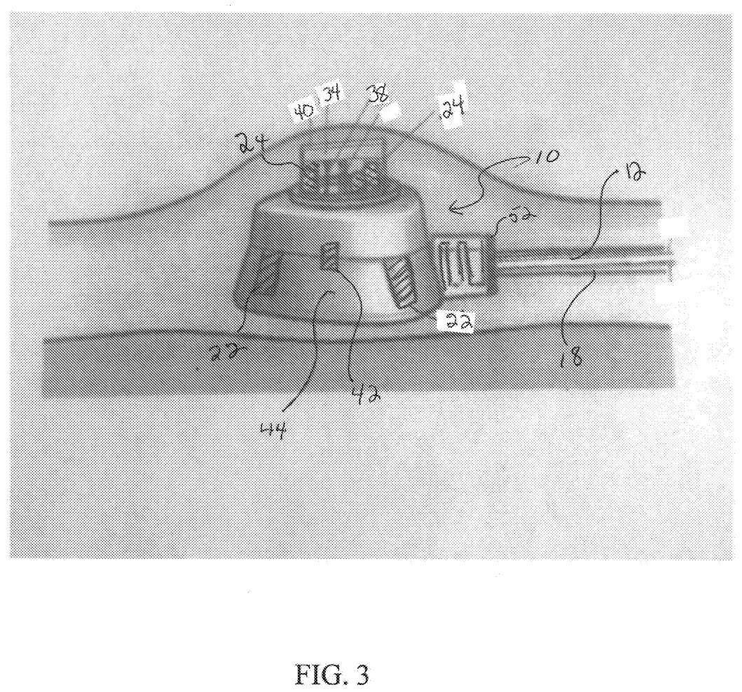

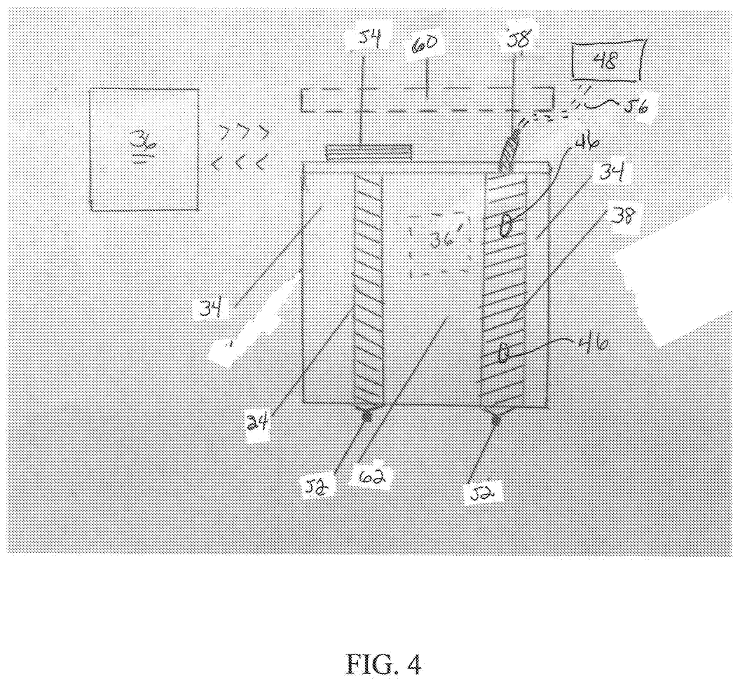

Referring generally to the figures, intravascular port 10 includes one or more access openings 22 configured to accept one or more sensors 32 and one or more treatment units 38 that are introduced through the skin and the membrane 30 by an introducer 17 and are configured to remain in place indefinitely. Alternatively, those of skill in the art will appreciate that the sensor 32, the treatment unit 38 or both may be removable and, therefore, replaceable. The one or more sensors 24 and treatment units 38 are configured to remain in the access openings 22 or may be advanced into the vasculature. The sensor 24 and or treatment unit 38 may be placed ex vivo and coupled to the access openings 22 or may be place in vivo directly into the access opening 22. The sensor 32 and treatment unit 38 may be coupled to a control unit 34 which is placed over the sensor 24 and treatment unit 38 as best seen in FIG. 3 or alternatively the sensor 32 and treatment unit 38 may be integrated into the control unit as best seen in FIG. 4. The control unit 34 may include a surface membrane that allows replenishment and/or replacement of the sensor 32 or treatment unit 38 or both. Optional clips 42 may be included on an outer surface 44 of the intravascular port that may be used to center the port in situ and maintain it in place.

The sensor 32 communicates with a processing unit 36 which may be located remotely or alternatively processing unit 36′ may be integrated with control unit 34. Processing unit 36 may include embedded microprocessors, digital signal processors, personal computers, laptop computers, notebook computers, palm top computers, network computers, Internet appliances, and processor-controlled devices configured to store data and software.

Processing unit 36 may include memory having a data base of knowledge including known normative data related to disease states. Processing unit 36 receives a signal from sensor which contains patient physiological parameters and analyzes the physiological parameters and other data obtained from the sensor 24. Processing unit 36 analyzes the data (i) in isolation as it is received; (ii) in the context of measurement and analysis based on the past history of the patient, which is stored in memory, or (iii) cross-references the patient data in the signal and cross-references it with the known normative data in the database. The processing unit 36 optionally includes a display device for displaying the output. The processing unit 36 can also include goal-directed therapies associated with particular disease states for providing suggested goal-directed treatments based on the cross-referencing step and outputs a suggested treatment by transmitting it wirelessly or by wire to a control unit 34 which may integrated with or be separate from the sensing or treatment system.

The control unit 34 is deployed through port membrane 30 and couples the sensor 24 and treatment unit 38. The control unit 34 may be placed over the port membrane 30 or may be remote from the intravascular port 10, may be deployed through the port membrane 30 or may be tethered to (via cables or catheters) or communicate wirelessly with the intravascular port 10, sensor 24 and treatment unit 38. Other ex vivo or in vivo sensing systems may be integrated with the intravascular port sensor 24 and treatment unit 38 The sensing may be in a fixed or adjustable cycle. The sensor may measure active or inactive analytes including but not limited to biochemical, hormonal, inflammatory, hematologic, genetic/nucleic acid and physiologic concentrations, as Well as vascular pressures, flow rates, pharmacologic concentrations, degradation products, pH, oxygen and carbon dioxide and toxic exposures. Sensing may also include qualitative features such as optical designs. The sensor 24 may be advanced via the blood vessel to distant sites and the sensor 24 deposited in the vessel or in organs. It also may be removed using the same system. The sensor 24 may be able to continually sense with a single sensor for extended periods or may have a single use cartridge which can be used as needed for intermittent measurements.

The treatment unit 38 includes one or more ports 46 thereon that are configured to infuse treatment based on, at least in part, the output from the processing unit. The treatment may include single or multiple infusions, continual or pulsatile of one or more active or inactive substances. Patient treatment is delivered by precision pumps or other infusion/injection devices 48 which may be attached to the port 10. The infusion devices 48 may use catheters or tubing 56 to infuse into the port chamber 16. The infusion devices 48 have an integrated receiver/energy source 62 to read and operate using data directly from the sensor or by receiving output from the processing unit 36 and/or commands from the control unit 34. The control unit 34 may also adjust the sensor's measurement cycle as well as the treatment parameters. The infusion device 48 may have refillable or disposable cartridges containing appropriate treatment infusions, pharmaceuticals and the like. In addition the treatment system may be integrated with other systems which may include but are not limited to anesthesia, respiratory, and cardiovascular control units such as pacemaker, anesthesia work station and respirator or other infusion systems such as via central or peripheral vein catheters. Multiple infusions may be used in a counterbalancing manner such as insulin/insulin analogs and glucagon/glucagon analogs/glucagon like substances/glucose to control sensed glucose concentrations. In. Other treatments not involving counterbalancing solutions may also be infused, such as but not limited to, vasopressors, bicarbonate, and blood/blood products. The treatment unit 38 may also incorporate surgical tools introduced via a port which can advance via the vessel to various organs to treat one or multiple organs with laser, infusion, injection, excision, or other techniques. The treatment unit 38 may also allow a separate device to be placed and deposited for use over time. It may then be removed if needed or may dissolve in place.

As best seer in FIG. 4, the control unit 34 may be integrated with the sensor 32 and the treatment unit 8. It may be a manually and/or automated system which allows adjustment of the sensor 32 and treatment units 38 based on the sensor data, transmitted data from other sources and/or programmable information. Processing unit 36 may be integrated with control unit 34 and includes algorithm 100 that may receive multiple inputs from the one or more sensors 24 to provide suggested goal-directed treatments. Control unit 36 may be integrated with a wireless system which can be monitored or controlled remotely. It may start/stop or adjust treatment modalities and cycles using learning and/or prefixed algorithms. It may monitor the status of the sensor 24 and treatment unit 38 to determine accuracy of delivery as well as replacement of components.

The sensor 32 can be placed through the skin and port membrane with passage into a vascular space to measure circulating analytes or other physiologic parameters. The sensor 32 may also be attached to a transmitter/energy source 62 placed on the port surface or the processing unit 36. This can send data to the treatment unit which may be attached to the same or separate port allowing closed or semi closed loop infusions. An open loop system is also within the scope of the invention.

FIG. 4 depicts an illustration of a typical control unit 34 The control 34 is implanted under the skin, on the surface of the port membrane 30 or in, the internal chamber 16. As depicted the sensor 32 and treatment unit 38 have been positioned in the control unit 34, or alternatively are integrated with the control unit 34. This may be accomplished during assembly of the control unit 34 or alternatively when the control unit 34 is placed wider the skin of the patient. The energy supply 62, hardware and transmitter for the control unit 34 are located centrally. The distal end of the sensor 32 and the distal end of the treatment unit 38 include a coupling element for coupling to a catheter that is positioned within the patient's vasculature. The sensor unit includes a sensor membrane 52 that may be used to replace and/or replenish the sensing requirements. The treatment unit 38 includes a coupling element 58 connected to tubing 56 for receiving an infusion of medications. An external system 58 may be placed in proximity or remotely to adjust and/or monitor the control unit 34.

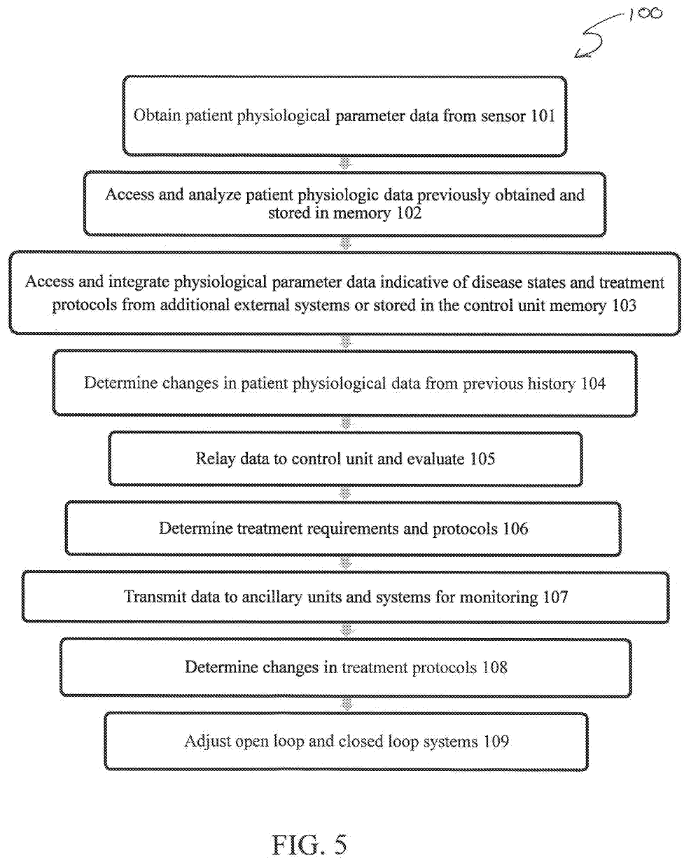

The method performed by the algorithm 100 included in the system of the present invention is depicted in FIG. 5. Patient physiological parameter data is obtained from sensor 101. Patient physiologic data previously obtained and stored in memory is accessed and analyzed 102. Optionally, physiological parameter data indicative of disease states and treatment protocols from additional external systems or stored in the control unit memory is accessed and integrated 103. Changes in patient physiological data from previous history is determined 104. Data is relayed to control unit and evaluated 105. Treatment requirements and protocols are determined 106. Data is optionally transmitted to ancillary units and systems for monitoring 107. Changes in treatment protocols may be determined 108. Open loop and closed loop systems are adjusted 109 to deliver therapy based on analysis.

While the invention has been described with reference to the specific embodiments thereof those skilled in the art will be able to make various modifications to the described embodiments of the invention without departing from the true spirit and scope of the invention. The terms and descriptions used herein are set forth by way of illustration only and are not meant as limitations. Those skilled in the art will recognize that these and other variations are possible within the spirit and scope of the invention as defined in the following claims and their equivalents.

What is claimed:

1. An intravascular port comprising:
a housing having an internal chamber and an outer body having a membrane sin a surface thereof, the housing including one or more access openings that communicate with the internal chamber;
one or more sensors received within the one or more access openings for sensing one or more physiological parameters of a patient;
one or more treatment units received within the internal chamber for providing a treatment to the patient;
a control unit in communication with the cane or more sensors and the one or more treatment units, the control unit configured to receive a signal from the one or more sensors and output a treatment protocol to the treatment unit for delivery to the patient.

2. The intravascular port of claim 1 wherein the control unit includes a processing device having a database of known data corresponding to various disease states, wherein the processing device is in operable communication with said one or more sensors and said database, said processing device configured to receive a signal from said one or more sensors and calculate a physiologic parameter of the patient therefrom, and cross-reference said patient physiologic parameter with the known data in the database and output a suggested treatment protocol to the treatment unit based on said cross-reference.

3. The intravascular port of claim 1 wherein the one or more sensors and the one or more treatment units are integrated with the control unit.

4. The intravascular port of claim 1 wherein the control unit is located remote from intravascular port.

5. The intravascular port of claim 2 wherein the treatment unit includes one or more treatment ports for delivery of medications.

6. The intravascular port of claim 1 further comprising an energy source for powering the control unit.

7. The intravascular port 6 wherein the energy source is positioned on an outer surface of the port.

8. The intravascular port of claim 1 wherein the one or more sensors include a membrane thereon for replenishment or replacement of the sensor.

9. The intravascular port of claim 1 further including a catheter coupled to the port, and implanted in the vasculature of a patient.

10. The intravascular port of claim 1 further including a plurality of clips on an outer surface thereof for centering and maintaining the intravascular port in position.

11. The intravascular port of claim 1 wherein the physiological parameters are selected from one or more of biochemical, hormonal, inflammatory, hematologic, genetic/nucleic acid, vascular pressures, flow rates, pharmacologic concentrations, degradation products, pH, oxygen, carbon, dioxide and toxic exposures.
